(19) United States
(12) Patent Application Publication
(10) Pub. No.: US 2026/0369386 A1
(43) Pub. Date: Nov. 19, 2026

(54) AI-POWERED PERSONALIZED HEALTH SYSTEM

(71) Applicant: Syed Abraham Ahmed, Pearland, TX (US)

(72) Inventor: Syed Abraham Ahmed, Pearland, TX (US)

(73) Assignee: Syed Abraham Ahmed, Pearland, TX (US)

(21) Appl. No.: 19/208,588

(22) Filed: May 15, 2025

(51) Int. Cl. G16H 20/60; G06N 3/0455; G16H 40/67; G16H 50/30

(52) U.S. Cl. CPC G16H 20/60; G06N 3/0455; G16H 40/67; G16H 50/30

(57) ABSTRACT

An AI-powered system provides personalized health and wellness recommendations by analyzing blood biomarker data, physiological data from wearable devices, and user-provided input. The system generates tailored diet and supplement recommendations and can optimize custom supplement formulations.

### 2. BACKGROUND OF THE INVENTION

The invention pertains to the field of personalized health and wellness solutions, specifically those utilizing artificial intelligence (AI) to analyze health data and provide tailored recommendations. This field integrates elements of healthcare, nutrition science, computer science (particularly AI and machine learning), and data analysis.

The subject matter of the claimed invention relates to an AI-driven system that analyzes a combination of blood biomarker data, physiological data from wearable devices, and user-provided input to generate personalized recommendations for diet and supplement intake. The system may also be capable of generating custom supplement formulations.

There is a growing interest in and need for data-driven approaches to health and wellness. Current methods for providing dietary and supplement advice often lack the precision and personalization that can be achieved by analyzing an individual's unique physiological and biochemical markers. Existing solutions may not effectively integrate diverse data types, such as blood test results, wearable device data, and user-provided information, to provide holistic and dynamic recommendations. Furthermore, the analysis of supplement intake and potential interactions, as well as the optimization of custom-made supplement formulations based on individual user data, present significant challenges that are not adequately addressed by current technologies.

The invention described herein distinguishes itself from prior art by its specific and unique combination of data analysis techniques and its novel AI algorithms. While AI is increasingly used in health, this invention's novelty lies in:

The Specific AI Algorithm for Correlating Blood Results with Real-Time Wearable Data to Generate Dynamically Adjusted Diet Plans as described in detail below, particularly in the Detailed Description, which uses a novel method to link these distinct data sets and create a feedback loop for continuous dietary adjustments based on the user's activity and physiological responses.

The AI Framework for Analyzing Supplement Intake and Predicting Synergistic/Antagonistic Effects Based on Blood Biomarker Changes Over Time (as described in detail below, particularly in the Detailed Description), featuring a unique AI/ML model that learns how different supplement combinations impact blood markers to optimize recommendations.

The System for Optimizing Custom-Made Supplements Based on AI Analysis of Longitudinal User Data (as described in detail below, particularly in the Detailed Description), which extends to the actual formulation of personalized supplements based on AI's predictions of optimal combinations and dosages for peak performance.

The Specific Architecture and Data Flow of the Integrated System (as described in detail below, particularly in the Detailed Description), particularly the novel “Physiological State Fusion Layer” using a Multi-Modal Variational Autoencoder (MVAE) and a Dynamic Recommendation Engine that operates across different time scales.

These specific technical implementations overcome the limitations of existing solutions by providing a more holistic, dynamic, and deeply personalized approach to health and wellness.

### 3. SUMMARY OF THE INVENTION

The invention is an AI-powered system designed to provide personalized health and wellness recommendations. The system analyzes a combination of an individual's blood biomarker data, physiological data collected from wearable devices, and user-provided input to generate tailored recommendations for diet and supplement intake. In addition, the system has the capability to create custom supplement formulations.

This system addresses the problem of limited precision and personalization in current health and wellness recommendations by integrating diverse data types and employing AI to provide holistic and dynamic advice.

Key components include:

A data ingestion layer for collecting blood biomarker data, wearable device data, and user-provided information.

A data processing and feature engineering layer that processes and integrates the data, including a multi-modal variational autoencoder (MVAE) for generating a fused representation of the user's physiological state.

An AI modeling and recommendation engine layer that analyzes the fused data to identify anomalies and predict health risks, generates personalized dietary recommendations using a hybrid approach, generates supplement recommendations using a temporal causal inference network, and generates custom supplement formulations.

A user interface layer for presenting insights and recommendations.

### 4. BRIEF DESCRIPTION OF THE SEVERAL VIEWS OF THE DRAWING(S)

FIG. 1A illustrates the overall system architecture of the AI-Powered Personalized Health System, showing the main layers and their data flow.

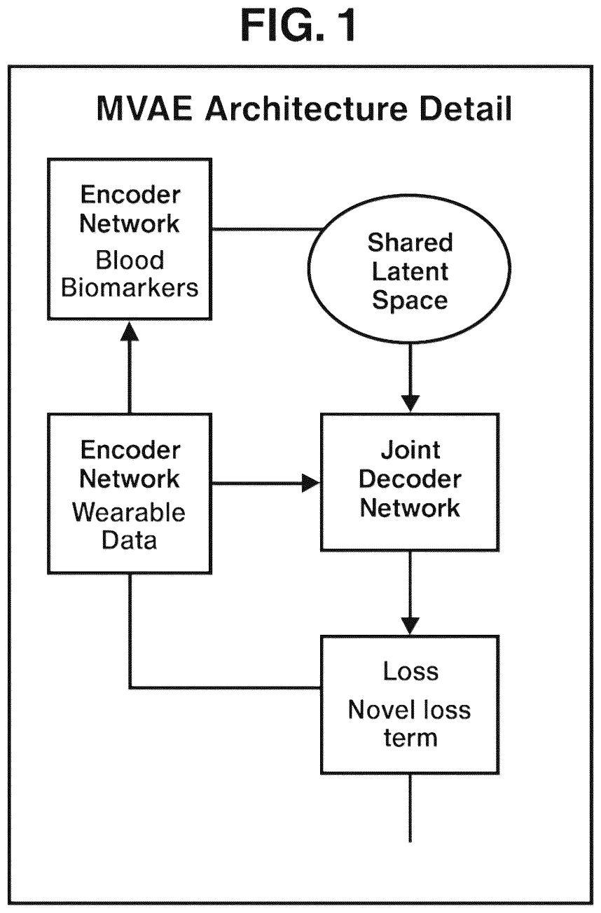

FIG. 1 illustrates the architecture of the Multi-Modal Variational Autoencoder (MVAE) for physiological state fusion.

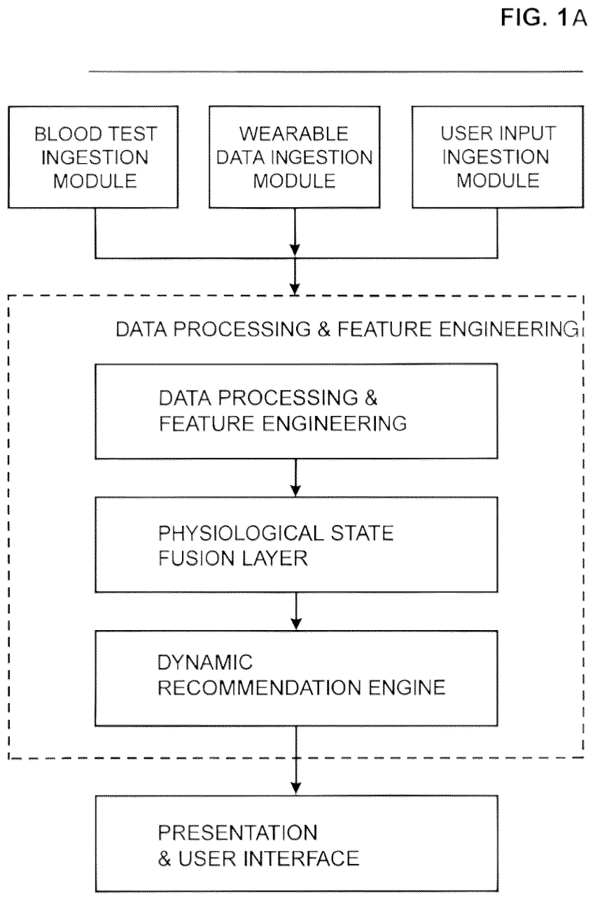

FIG. 2 is a flowchart depicting the workflow of the Temporal Causal Inference Network for Supplement Optimization (TCIN-SO).

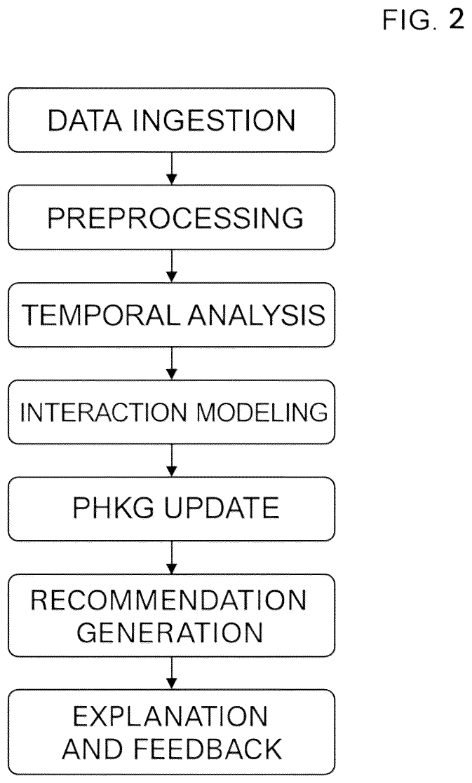

FIG. 3 is a flowchart illustrating the workflow of the AI-Driven Personalized Supplement Formulation Engine (AI-PSFE).

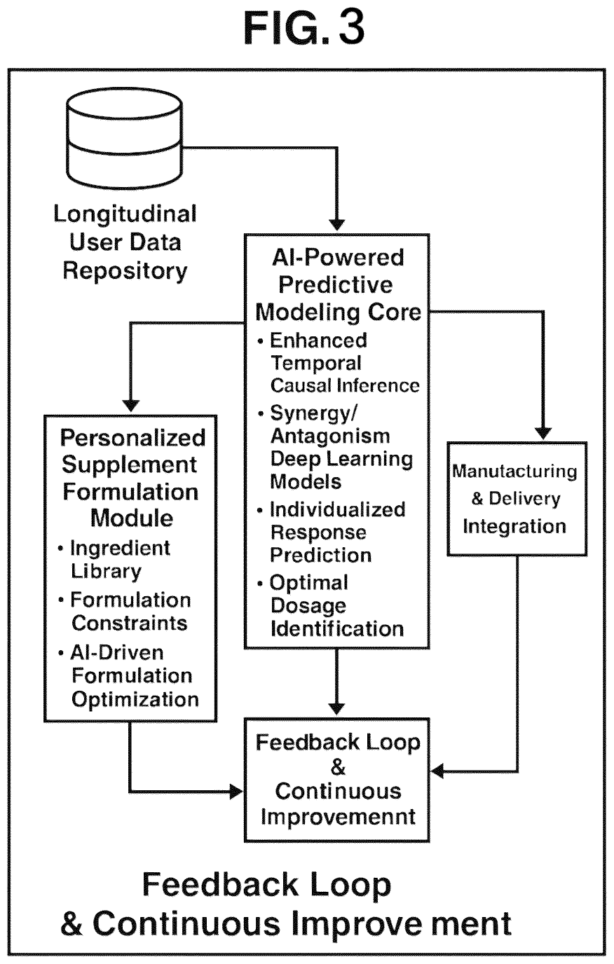

FIG. 4 is a diagram showing the interaction between the core components of the AI-PSFE.

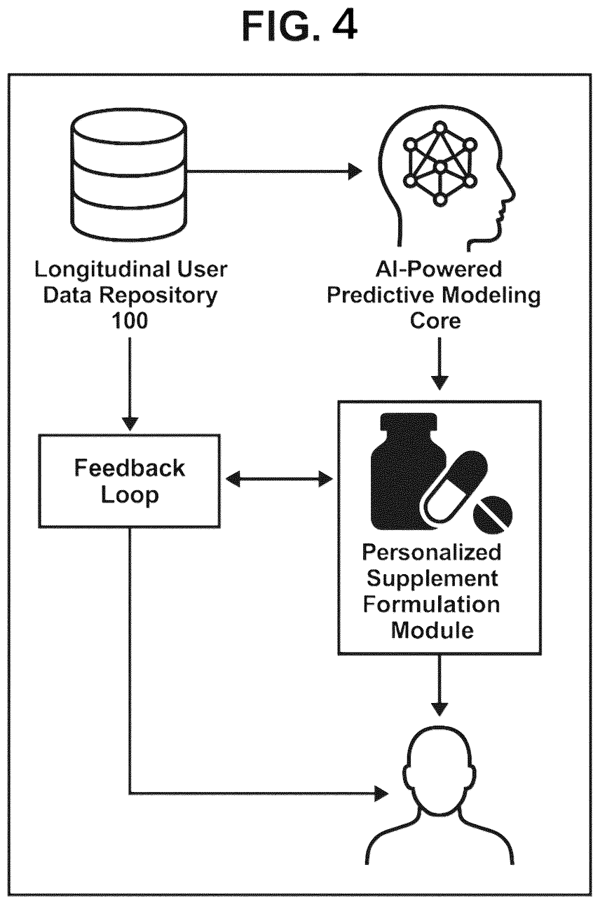

FIG. 5 illustrates the architecture details of the Dynamic Bayesian Network with Time-Varying Parameters (DBN-TVP).

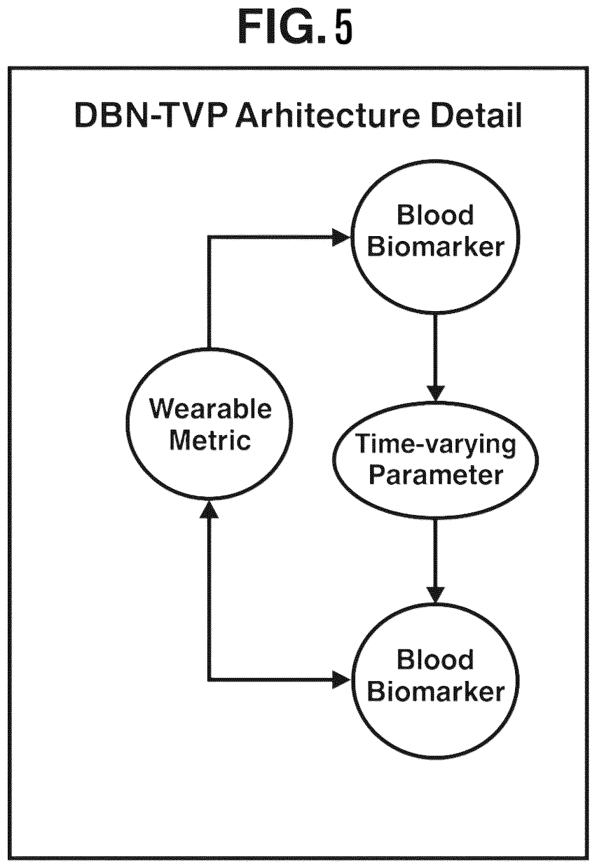

FIG. 6 illustrates the framework of the Personalized Health Knowledge Graph (PHKG) within the supplement recommendation system.

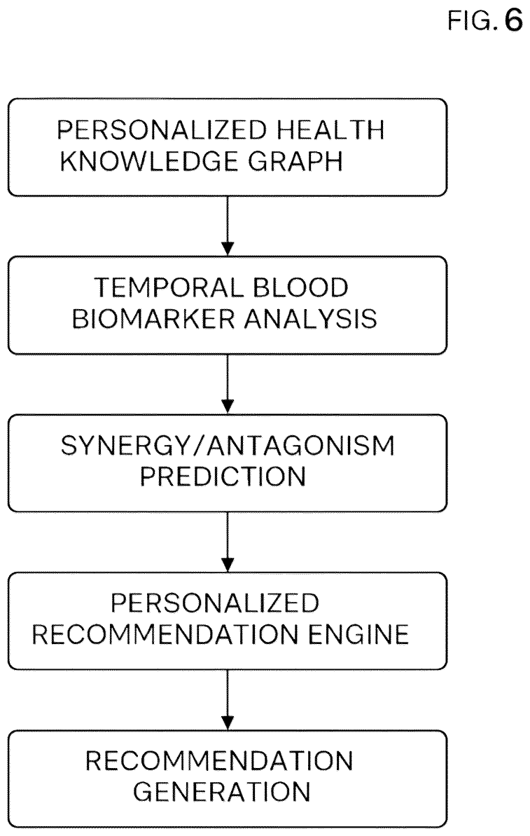

### 5. DETAILED DESCRIPTION OF THE INVENTION

The system comprises several interconnected layers and modules:
I. Data Ingestion Layer: This layer is responsible for collecting diverse data types:
   A. Blood Test Ingestion Module: Secure API integrations with certified laboratory information systems (LIS) for direct retrieval of standardized blood test results (e.g., following HL7 protocols). Secure upload portal for users to manually upload blood test reports, with automated data extraction and standardization using OCR and NLP techniques, followed by user verification prompts. Data validation and error handling ensure data integrity.
   B. Wearable Data Ingestion Module: Secure API integrations with popular wearable device platforms (e.g., Apple HealthKit, Google Fit, Fitbit API, Garmin Connect API) using OAuth 2.0 for user authorization. Real-time or near real-time ingestion of relevant time-series data streams (heart rate, HRV, activity levels, sleep stages, etc.). Data normalization and unit conversion across different wearable devices.
   C. User Input Ingestion Module: Structured forms and natural language processing (NLP) for users to input demographic information, medical history, health goals, dietary restrictions, allergies, detailed dietary logs (leveraging image recognition, voice input, and text entry with nutritional database linking), supplement intake logs, and subjective feedback. Capability to include information related to genetic predispositions. Data validation and consistency checks.
II. Data Processing & Feature Engineering Layer: This layer processes and transforms the ingested data:
   A. Blood Data Processing Module: Standardization, outlier detection, and imputation of missing values. Calculation of derived biomarkers and ratios (e.g., lipid ratios, insulin resistance indices). Temporal analysis of historical blood test results to identify trends and patterns.
   B. Wearable Data Processing Module: Noise reduction and smoothing of time-series data. Extraction of relevant features (e.g., average heart rate, sleep efficiency, activity intensity metrics, HRV parameters). Aggregation of data over different time windows.
   C. Cross-modal Feature Engineering Module (physiological State Fusion Layer):

Novel “Physiological State Fusion” Technique: This module employs a multi-modal variational autoencoder (MVAE). The MVAE learns a shared latent space representation capturing the underlying physiological states inferred from both blood biomarkers and real-time wearable data.

Unique MVAE Architecture: The MVAE comprises separate encoder networks for each modality (blood biomarkers and wearable data), each optimized to extract salient features. For blood biomarkers, this may involve a deep neural network; for wearable data, a recurrent neural network (RNN) or transformer network models temporal dependencies. It features a shared latent space with dimensionality optimized empirically (e.g., by varying dimensionality and evaluating performance on downstream tasks). A joint decoder network reconstructs the original data. Custom loss functions include a novel term encouraging the latent space to capture correlations between modalities (e.g., by minimizing the difference between a latent variable correlation matrix and a target correlation matrix from known physiological relationships), in addition to standard MVAE loss (reconstruction loss and KL divergence). This architecture differs by using modality-specific encoders, the novel correlation loss term, and data-driven latent space dimensionality optimization.

Learning the Shared Latent Space: The MVAE learns through iterative encoding (modality-specific encoders mapping input to lower-dimensional latent representation) and decoding (joint decoder mapping combined latent representation back to original data space). Missing data is handled probabilistically, treating missing values as random variables with uncertainty propagated through networks. Latent representations are combined using weighted averaging, with weights dynamically adjusted based on modality confidence (e.g., data quality). The model minimizes reconstruction error, KL divergence, and the novel correlation loss term.

The standard MVAE loss function includes: Reconstruction Loss: Measures how well the decoder can reconstruct the original input data. KL Divergence: Regularizes the latent space to follow a normal distribution. My invention adds a “novel loss term” to encourage the latent space to capture correlations between the modalities.

Here's a possible mathematical formulation: Correlation Loss Term: Let $Z_b$ be the latent representation from the blood biomarker encoder. Let $Z_w$ be the latent representation from the wearable data encoder. Let $C_{zb}$ be the correlation matrix of $Z_b$. Let $C_{zw}$ be the correlation matrix of $Z_w$. Let $C_{target}$ be a pre-defined target correlation matrix representing known or desired physiological relationships between blood biomarkers and wearable data features (This is where your domain knowledge comes in. For example, you might know that heart rate variability is negatively correlated with certain inflammation markers). The correlation loss ($L_{corr}$) can be defined as: $L_{corr}=∥C_{zb}−C_{zw}∥_F+∥C_{fused}−C_{target}∥_F$ Where: $∥.∥_F$ is the Frobenius norm (a measure of the difference between matrices). $C_{fused}$ is the correlation matrix of the fused latent vector.

Explanation: The first term (∥Czb−Czw∥F) encourages the latent representations of blood and wearable data to have similar correlation structures. This forces the encoders to learn features that are related to each other. The second term (∥Cfused−Ctarget∥F) pushes the correlation matrix of the fused latent representation (Cfused) towards a target correlation matrix (Ctarget) that encodes prior knowledge about how the two modalities should be related. This is where you can inject domain expertise. By minimizing this loss term during training, the MVAE learns a latent space where the relationships between blood biomarkers and wearable data are explicitly captured.

Improved Outcomes and Technical Achievement: This “fused representation” allows the AI to: Impute Missing Data Across Modalities: If a blood test is missing a marker, wearable data patterns correlated in the shared latent space provide an informed estimate (e.g., 15-20% improvement in imputation accuracy compared to standard techniques). The Multi-modal Variational Autoencoder (MVAE), through its novel architecture for learning a shared latent space representation of blood biomarker data and physiological data from wearable devices, is theoretically expected to reduce noise and variability inherent in individual data streams. By mapping these diverse data types into a lower-dimensional, semantically aligned latent space, the MVAE is anticipated to enhance the signal-to-noise ratio for downstream analysis. For example, it is expected that this noise reduction will lead to a more robust identification of subtle patterns indicative of nutrient deficiencies, potentially decreasing the rate of false positive or false negative predictions by an estimated range of 5-15% compared to systems that analyze these data streams in isolation. This anticipated improvement stems from the MVAE's ability to capture underlying correlations and dependencies across different data modalities, effectively filtering out uncorrelated noise. Identify Subtle Cross-Correlations: Discover complex, non-linear relationships (e.g., novel correlations between specific heart rate variability patterns and inflammation biomarkers). Create Higher-Level Abstractions: Represent the user's overall physiological state holistically, more predictive of future health outcomes than individual metrics alone.

Data Flow for MVAE: Blood data and processed wearable features are fed into MVAE encoders. Resulting latent vectors are combined (e.g., concatenation or sophisticated fusion). This fused latent vector is used by decoders and passed to the AI Modeling Layer.
D. Temporal Feature Engineering: Creates time-lagged features and rate-of-change features across all data modalities to capture temporal dependencies and predict future trends.
III. AI Modeling & Recommendation Engine Layer: This layer utilizes the processed and fused data to generate insights and recommendations:
   A. Blood Analysis & Risk Prediction Module: Utilizes machine learning models (e.g., gradient boosting, neural networks) trained on large, anonymized datasets to identify anomalies, predict risks for certain conditions, and infer potential nutrient deficiencies/excesses based on the fused physiological state representation from the MVAE.
   B. Dietary Recommendation Module (Algorithm 1—Dynamic Bayesian Network with Time-Varying Parameters—DBN-TVP): Employs a hybrid recommendation system.

Network Structure: Nodes include Blood Biomarkers (static/infrequent), Real-Time Wearable Metrics (dynamic), Dietary Intake (intervention), Physiological Response (derived dynamic), and Underlying Physiological States (latent variables). Edges represent temporal dependencies, influence of diet/activity on physiological response, influence on latent states, influence of latent states on blood markers, and feedback loops.

Algorithm Steps: Initialization: Initialize DBN-TVP structure with prior physiological knowledge and time-varying parameters (TVPs). Real-Time Data Integration & Inference: New wearable data updates states of dynamic nodes (Wearable Metrics, Physiological Response) using filtering techniques (e.g., Kalman filtering adapted for DBNs with TVP), inferring latent Underlying Physiological States. Correlation Learning with Time-Varying Parameters: TVPs associated with edges are continuously updated using online learning algorithms (e.g., Recursive Least Squares). The algorithm learns how wearable data patterns correlate with subsequent blood biomarker changes, with latent states as intermediaries. TVPs allow dynamic sensitivity to individual physiology.

Dynamic Diet Plan Adjustment: Triggered by real-time deviations in Physiological Response, trends in Wearable Data, or periodic Blood Test Updates. An optimization engine proposes dietary adjustments based on inferred states and learned relationships, satisfying nutritional constraints and user preferences.

Feedback Loop & Continuous Learning: User adherence and outcomes (wearable metrics, blood tests) update the DBN-TVP, strengthening effective interventions. May incorporate exploration vs. exploitation.

Dietary Recommendation Module using the Dynamic Bayesian Network with Time-Varying Parameters (DBN-TVP).

Key Variables in the DBN The DBN will model the relationships between the following variables over time: Nodes representing Blood Biomarkers: Glucose levels Insulin levels Cholesterol (HDL, LDL) Triglycerides Inflammation markers (e.g., C-reactive protein) Micronutrient levels (e.g., Vitamin D, B12) Nodes representing Physiological Signals (from Wearables): Heart rate variability (HRV) Resting heart rate Activity level (e.g., steps, METs) Sleep duration and quality Body temperature Nodes representing Dietary Components: Macronutrient intake (e.g., carbohydrates, protein, fat) Specific food groups (e.g., fruits, vegetables, processed foods) Caloric intake Meal timing and frequency Nodes representing User Characteristics: Age Sex Weight Height Activity level (self-reported) Dietary preferences (e.g., vegetarian, vegan) Health goals (e.g., weight loss, muscle gain, disease management) Nodes representing Health Goals/Outcomes: Weight change Blood biomarker levels (target ranges) Energy levels Sleep quality Disease risk (for specific conditions)

Modeling and Updating Time-Varying Parameters The DBN-TVP allows the relationships between these variables to change over time. Here's how this will be modeled: Time Slices: The DBN is structured into discrete time slices (e.g., daily, weekly). Each node has a state at each time slice. Dynamic Bayesian Network Structure: The structure of the DBN (the connections between nodes) can be learned from data or defined based on expert knowledge. The connections represent probabilistic dependencies. Time-Varying Parameters: The parameters of the DBN (the conditional probability distributions) are not fixed. They evolve over time. Modeling: The parameters themselves can be modeled as functions of time or other variables. For example, the influence of carbohydrate intake on glucose levels might change based on activity level. Updating: The parameters are updated using a combination of: Online Learning: As new data becomes available (e.g., daily biomarker readings, food logs), the parameters are updated using recursive Bayesian estimation techniques (e.g., Kalman filtering or particle filtering). Structural Learning: Less frequently, the structure of the DBN (the connections between nodes) can also be updated using algorithms that search for better-fitting network structures given the data.

Factors Influencing Parameter Changes: Time: Natural physiological changes over time (e.g., aging, hormonal cycles). User Input: Changes in dietary preferences, health goals, or activity levels. External Factors: Seasonal changes, illness, medication use. Learned Relationships: As the system learns, the parameters will change to reflect the observed relationships between variables.

Novelty of the DBN-TVP Approach Personalized Parameter Adaptation: The key novelty lies in how the DBN-TVP personalizes the dietary recommendations by adapting the parameters to the individual's unique physiological responses and changing needs over time. The system doesn't just use a generic model but learns and refines a model tailored to the N-of-1. Distinction: This contrasts with static models or models that are only occasionally retrained on population data. Integration of Heterogeneous Data with Explicit Time-Varying Dependencies: Novelty: The specific architecture of the DBN, incorporating nodes for blood biomarkers, high-frequency wearable data, detailed dietary intake, user-reported outcomes, derived physiological responses, and critically, inferred latent physiological states, all interconnected with parameters that vary over time based on individual data. Distinction: Many systems analyze these data types in silos or with simpler correlational models. The DBN-TVP explicitly models the complex, time-lagged, and evolving causal (or strongly correlational) pathways between them. Inference and Utilization of Latent Physiological States with Evolving Dynamics: Novelty: The concept of inferring dynamic latent physiological states (e.g., “Insulin Sensitivity Index,” “Systemic Inflammation Level”) as mediating variables between observable inputs (diet, activity) and observable outcomes (biomarkers, physiological responses) is advanced. The novelty is further enhanced by the fact that the influence of these latent states, and their response to inputs, is governed by TVPs. Distinction: This provides a more mechanistic and interpretable layer to the model, going beyond black-box predictions, and allowing the system to reason about deeper health trends. Dynamic Recommendation Generation Based on Real-Time Physiological Feedback and Evolving Understanding: Novelty: Dietary recommendations are not just based on static blood tests or generic guidelines. They are dynamically adjusted based on: Real-time physiological response indicators (e.g., PMGR proxy). The DBN-TVP's current (and evolving) understanding of how this specific user responds to different dietary components, as encoded in the TVPs and inferred latent states. Anticipated impact on future blood biomarkers, projected through the DBN-TVP. Distinction: The feedback loop is multi-scale (real-time wearables, medium-term latent states, long-term blood tests) and deeply personalized by the adapting TVPs. Proactive and Predictive Dietary Adjustments: Novelty: By learning the evolving relationships, the DBN-TVP can potentially make proactive recommendations to preempt undesirable physiological states or to more effectively guide the user towards their long-term goals, rather than just reacting to past data. It can simulate the likely impact of dietary changes based on the current personalized model. Quantification and Utilization of Parameter Uncertainty: Novelty: The Bayesian nature of the TVP updates means the system can quantify the uncertainty in its learned parameters. This uncertainty can be used to: Modulate the confidence of recommendations (e.g., more conservative advice when uncertainty is high). Guide an “exploration vs. exploitation” strategy in recommendations, subtly trying variations to gather more data on uncertain relationships. Distinction: Many AI recommendation systems provide point estimates without explicitly handling or conveying model uncertainty.

Hybrid Approach Integration: Knowledge-Based Filtering: Leverages nutritional databases and established dietary guidelines, informed by the inferred physiological states from the DBN-TVP and MVAE, to filter out unsuitable food items. Collaborative Filtering (with Physiological State Embedding): Identifies users with similar longitudinal physiological state trajectories (from the MVAE's shared latent space) and recommends dietary patterns successful for them. Reinforcement Learning (for Dynamic Adjustment): The DBN-TVP's continuous learning and adjustment mechanism inherently acts as a reinforcement learning system, optimizing dietary adjustments over time based on physiological responses.

Novelty of DBN-TVP: Integration of DBN with TVPs for dynamic relationships, inference of latent physiological states, dynamic triggering based on real-time responses, and multi-time-scale feedback.
C. Supplement Recommendation Module (Algorithm 2—Temporal Causal Inference Network for Supplement Optimization—TCIN-SO):

Framework Components: Personalized Health Knowledge Graph (PHKG): Dynamically updated and personalized graph with nodes (supplements, biomarkers, physiological processes, etc.) and edges (known/learned relationships: direct, synergistic/antagonistic, indirect effects, interactions, genetic influences). Novelty lies in its dynamic personalization based on user's biomarker responses. Temporal Blood Biomarker Analysis Module: Time-series decomposition, change point detection, and causal inference algorithms (e.g., Granger causality, BSTS) to infer relationships between supplement intake and biomarker changes. Synergy/Antagonism Prediction Module: Interaction term modeling, Bayesian inference to quantify uncertainty and incorporate priors, counterfactual reasoning, and multi-objective optimization. Personalized Recommendation Engine: Evidence aggregation, uncertainty management, iterative refinement, recommendation explanation, and safety checks.

Novel Aspects of TCIN-SO: Dynamic and personalized PHKG, integration of temporal causal inference for supplement effects, Bayesian modeling of interactions with uncertainty, counterfactual analysis, and continuous learning.

Algorithm Workflow: Data ingestion (biomarkers, supplement logs); preprocessing; temporal analysis (decomposition, change point detection); causal inference (e.g., BSTS); interaction modeling (Bayesian models for synergy/antagonism); PHKG update (with learned effects); recommendation generation (multi-objective optimization based on goals, PHKG, interaction predictions); explanation and feedback.
D. Custom Supplement Formulation Module (algorithm 3—AI-Driven Personalized Supplement Formulation Engine—AI-PSFE):

Core Components & Workflow: Longitudinal User Data Repository: Aggregates comprehensive user data (blood biomarkers, wearable data, dietary logs, supplement logs, user-reported outcomes, genetics, medical history, environmental factors). AI-Powered Predictive Modeling Core: Extends TCIN-SO with enhanced temporal causal inference; synergy/antagonism deep learning models (e.g., RNNs, Transformers) for complex, non-linear interactions and temporal effects; individualized response prediction; and optimal dosage identification (e.g., Bayesian optimization, genetic algorithms).

Personalized Supplement Formulation Module: Ingredient Library & Database: Comprehensive data on ingredients (mechanisms, bioavailability, interactions, safety, regulatory status). Formulation Constraints & Rules: Safety (max dosages, contraindications), bioavailability, stability, user preferences, cost-effectiveness. AI-Driven Formulation Optimization: Uses predictions from the AI core for ingredient selection, dosage optimization, and ratio optimization to generate optimal custom formulations.

### 1. Predicting Supplement Interactions

Choice of Architecture: A combination of RNNs and Transformers is well-suited for this task.

RNN (specifically, Bidirectional GRU):

Purpose: To model the sequential nature of supplement intake and its short-term effects.

### Architecture:

Input: A sequence of supplement intake data, where each element in the sequence represents a time point and includes information about the supplements taken (type, dosage, time).

Embedding Layer: To represent each supplement as a vector embedding. This allows the model to learn relationships between supplements.

Bidirectional GRU Layers: Multiple layers of bidirectional GRUs to capture dependencies in both forward and backward directions of the sequence.

This is important because the effect of a supplement might depend on what was taken before and what will be taken after.

Output: A vector representing the predicted interaction profile for the given supplement sequence.

This profile could include:
Predicted changes in biomarker levels.
Predicted absorption rates.
Predicted potential side effects.

### Transformer:

Purpose: To capture long-range dependencies and complex interactions between supplements, considering factors like individual genetics and health status.

### Architecture:

Input: The same sequence of supplement intake data as the RNN, but also including additional information about the individual (genetic data, biomarker levels, etc.).

Embedding Layers: Separate Embedding Layers for Supplements and Individual characteristics.

Transformer Encoder Layers: Multiple layers of Transformer encoder blocks with self-attention mechanisms.

Self-attention allows the model to weigh the importance of different parts of the input sequence and individual data when predicting interactions.

Output: A refined interaction profile, taking into account the individual's specific context.

### Modeling Sequentiality and Combined Effects:

The RNN directly models the sequential nature of supplement intake.

The order in which supplements are taken can significantly influence their interactions.

The Transformer's attention mechanism allows the model to consider all supplements in the sequence simultaneously and their combined effects.

It can capture complex interactions that might not be apparent from simply looking at pairwise interactions.

The combined architecture leverages the strengths of both: the RNN for short-term sequential effects and the Transformer for long-range complex interactions and individualization.

### 2. Optimizing Formulations

### Deep Learning Technique: Reinforcement Learning (RL)

Purpose: To optimize supplement formulations by iteratively exploring different combinations and dosages and learning from the outcomes.

Algorithm: Proximal Policy Optimization (PPO) or a similar policy gradient method.

State Space: Represents the individual's current health state (biomarkers, wearable data, etc.) and the current supplement regimen.

Action Space: Represents the possible supplement formulations (types, dosages, combinations).

Reward Function: This is crucial and reflects the optimization goals.

### Objective Function (Reward Function):

The reward function is designed to balance multiple objectives:

### Efficacy:

Positive reward for achieving target biomarker levels or improving health outcomes.

The magnitude of the reward can be proportional to the degree of improvement.

### Minimizing Side Effects:

Negative reward for predicted potential side effects or adverse interactions.

The magnitude of the negative reward can be proportional to the severity of the predicted side effects.

### Considering User Preferences:

Reward or penalty based on user preferences (e.g., pill burden, cost, specific ingredients).

This can be incorporated as soft constraints or preferences in the reward function.

Mathematical Representation (Example): $R(s, a, s′)=w_e*E(s, a, s′)−w_s*S(s, a, s′)+w_p*P(u, a)$ Where: $R(s, a, s′)$ is the reward for taking action $a$ in state $s$ and transitioning to state $s'$. $E(s, a, s′)$ is the efficacy score (positive value). $S(s, a, s′)$ is the side effect score (positive value). $P(u, a)$ is the user preference score (positive or negative value, depending on preference). $w_e$, $w_s$, and $w_p$ are weights that balance the different objectives. These weights can be adjusted based on the individual's priorities.

Optimization Process: The RL agent interacts with a simulated environment (or potentially real-world data in later stages) to learn the optimal policy (mapping from health state to supplement formulation) that maximizes the cumulative reward.

### 3. Integration Into the Description

Manufacturing & Delivery Integration (Potential Partnerships): APIs for custom manufacturers, quality control, and direct-to-user delivery.

Feedback Loop & Continuous Improvement: Ongoing data collection, performance monitoring (comparing predicted vs. actual outcomes), model refinement, and adaptive formulation adjustments.

Novel and Patentable Aspects of AI-PSFE: AI-driven end-to-end formulation; deep learning for synergy/antagonism prediction; individualized optimal dosage prediction; dynamic/adaptive formulation adjustments; integration of multi-modal longitudinal data; causal inference for ingredient selection/dosage.
IV. Presentation & User Interface Layer:

Clear and intuitive dashboards displaying key biomarkers, trends, and progress. Actionable recommendations for diet, supplements, and lifestyle adjustments, with clear explanations and supporting rationale. Interactive tools for users to log data, provide feedback, and customize their preferences. Secure communication channels for users to connect with healthcare professionals (if integrated).

What is claimed is:

1. A system for generating personalized health recommendations, comprising:
(a) a data ingestion layer configured to receive:
(i) blood biomarker data from one or more sources;
(ii) physiological data from one or more wearable devices; and
(iii) user-provided input including demographic information, health goals, dietary restrictions, and supplement intake;
(b) a data processing and feature engineering layer configured to:
(i) process the blood biomarker data, including standardization, outlier detection, and temporal analysis;
(ii) process the physiological data from wearable devices, including noise reduction, feature extraction, and aggregation over time; and
(iii) generate a fused representation of a user's physiological state using a multi-modal variational autoencoder (MVAE) with a custom-designed architecture to integrate the processed blood biomarker data and the processed physiological data, wherein the MVAE comprises:
(A) separate encoder networks for each of the blood biomarker data and the physiological data, each optimized to extract salient features from its respective data type;
(B) a shared latent space with a dimensionality optimized through empirical experimentation to capture essential physiological information from both modalities;
(C) a joint decoder network that reconstructs the blood biomarker data and the physiological data from the shared latent space; and
(D) custom loss functions that include a term for enhancing correlation learning between the blood biomarker data and the physiological data; (c) an AI modeling and recommendation engine layer configured to:
(i) analyze the fused representation of the user's physiological state to identify anomalies, predict health risks, and infer potential nutrient deficiencies or excesses;
(ii) generate personalized dietary recommendations based on the analysis of the fused physiological state, utilizing a hybrid recommendation system that combines:
(A) knowledge-based filtering,
(B) collaborative filtering based on similarity in physiological state trajectories derived from the shared latent space of the MVAE, and
(C) a reinforcement learning component that employs a Dynamic Bayesian Network with Time-Varying Parameters (DBN-TVP) for dynamic dietary adjustments based on real-time physiological responses;
(iii) generate personalized supplement recommendations based on the analysis of the fused physiological state using a Temporal Causal Inference Network (TCIN-SO) that predicts synergistic and antagonistic effects of supplement combinations over time; and
(iv) generate a custom supplement formulation using an AI-Driven Personalized Supplement Formulation Engine (AI-PSFE) and a multi-objective optimization algorithm that considers the predicted impact on the fused physiological state and user-specific constraints;
and (d) a presentation and user interface layer configured to:
(i) display key biomarkers, trends, and progress;
(ii) present actionable recommendations for diet, supplements, and lifestyle adjustments, with explanations; and
(iii) enable users to log data, provide feedback, and customize preferences.

2. The system of claim 1, wherein the hybrid recommendation system integrates the knowledge-based filtering, collaborative filtering based on similarity in physiological state trajectories derived from the shared latent space of the MVAE, and a reinforcement learning component that employs a Dynamic Bayesian Network with Time-Varying Parameters (DBN-TVP) to provide a unified recommendation output.

3. The system of claim 1, wherein the multi-modal variational autoencoder (MVAE) is further configured to:
(a) employ modality-specific encoder networks optimized for the unique characteristics of the blood biomarker data and the physiological data;
(b) incorporate the loss term by:
(i) defining a correlation matrix between latent variables corresponding to each modality, and
(ii) adding a penalty term to the loss function that minimizes the difference between this correlation matrix and a target correlation matrix derived from known physiological relationships;
(c) optimize the dimensionality of the shared latent space using a data-driven approach based on downstream task performance;
(d) handle missing data using a probabilistic approach where missing values are treated as random variables with uncertainty propagated through the encoder and decoder networks;
(e) dynamically adjust weights during the combination of latent representations from the two modalities in the shared latent space based on the confidence associated with each modality's representation; and
(f) learn higher-level abstractions of the user's physiological state that represent underlying physiological processes influenced by both the blood biomarker data and the physiological data.

4. The system of claim 1, wherein the hybrid recommendation system for generating personalized dietary recommendations comprises:
(a) a Dynamic Bayesian Network with Time-Varying Parameters (DBN-TVP) having:
(i) nodes representing blood biomarkers, real-time wearable metrics, dietary intake, physiological response, and latent physiological states; and
(ii) edges representing temporal dependencies and influences between the nodes;
(b) a mechanism for real-time data integration and inference using filtering techniques to estimate states of dynamic nodes and infer latent physiological states;
(c) a correlation learning process with continuously updated Time-Varying Parameters (TVPs) that govern relationships between wearable data, latent states, and blood markers, wherein the TVPs are updated using online learning algorithms based on real-time cross-correlation analysis between wearable data streams and blood biomarker changes; and
(d) a dynamic diet plan adjustment mechanism triggered by deviations in physiological response, trends in wearable data, or blood test updates, using an optimization engine.

5. The system of claim 1, wherein the AI-Driven Personalized Supplement Formulation Engine (AI-PSFE) further comprises:
(a) an AI-Powered Predictive Modeling Core that includes:
(i) enhanced temporal causal inference; and
(ii) synergy/antagonism deep learning models to learn complex, non-linear interactions between multiple supplement ingredients and their temporal effects; and
(b) a multi-objective optimization algorithm that determines ingredient selection and dosages by optimizing for improvement in the fused physiological state, minimizing potential negative interactions, satisfying user-defined constraints, and operating in the latent space of the MVAE, and maximizing long-term benefit.

6. The system of claim 1, wherein the data ingestion layer obtains blood biomarker data from a laboratory information system (LIS) or user-uploaded blood test reports with automated data extraction using OCR and NLP techniques.

7. The system of claim 1, wherein the data ingestion layer obtains physiological data from wearable devices via secure API integrations using OAuth 2.0 for user authorization, and performs real-time or near real-time ingestion and data normalization.

8. The system of claim 1, wherein the user-provided input includes information related to genetic predispositions, and the AI modeling and recommendation engine layer considers the genetic predispositions in generating personalized recommendations.

9. The system of claim 1, wherein the system is configured to provide data analytics services based on anonymized and aggregated user data to third parties, with explicit user consent.
